(19) United States
(12) Patent Application Publication
(10) Pub. No.: US 2003/0104463 A1
(43) Pub. Date: Jun. 5, 2003

(54) Identification of pharmaceutical targets

(75) Inventors: Bernd Schuermann, Haimhausen (DE); Martin Stetter, Munchen (DE)

Correspondence Address: STAAS & HALSEY LLP, 700 11TH STREET, NW, SUITE 500, WASHINGTON, DC, 20001, US

(73) Assignee: Siemens Aktiengesellschaft, Munich (DE)

(21) Appl. No.: 10/307,997

(22) Filed: Dec. 3, 2002

(30) Foreign Application Priority Data
Dec. 3, 2001 (DE) 101 59262.0

(51) Int. Cl. C12Q 1/68; G06F 19/00; G01N 33/48; G01N 33/50

(52) U.S. Cl. 435/6; 702/20

(57) ABSTRACT

In order to identify pharmaceutical targets, at least one correlation between the expression rates of different genes of a cell is ascertained by evaluating a plurality of gene expression patterns. In this case, correlations of second or higher order are considered. The correlations make it possible to infer causal relationships between different genes and the associated proteins. The regulatory network of the cell being studied can be therefore deduced from the correlations. Suitable targets can be identified from the regulatory network which has been deduced in such a way.

### CROSS REFERENCE TO RELATED APPLICATIONS

This application is based on and hereby claims priority to German Application No. 101 59 262.0 filed on Dec. 3, 2001, the contents of which are hereby incorporated by reference.

### BACKGROUND OF THE INVENTION

The human genome comprises approximately 20,000 to 80,000 genes, which contain the genetic code for about one million proteins. In the specialized cells of the body, only subsets of the total number of genes are actually read (expressed) in each case. Taken together, the proteins produced in this way are referred to as the proteome of this cell. The mutual interaction of the proteins, as well as their interaction with the DNA, represents the most important part of the mechanism governing the development of the human body-from the fertilized ovum, as well as all the bodily functions. In terms of information technology, the genome therefore represents a procedural code for the structure and function of the human body.

Many diseases and dysfunctions of the body are due to problems with the functional network made up of the genome and the proteome. Therefore, some medications act as agonists or antagonists for specific target proteins, that is to say they increase or decrease the function of a protein, with the aim of returning the network formed by the proteome and genome to a normal mode of function. These target proteins have to date been derived according to heuristic principles from biochemical considerations. It is in this case often unclear whether the dysfunction of a protein actually represents the cause of the disease, or whether it only represents one of the symptoms of a concealed misregulation at another point of the network.

For the development of improved therapies, therefore, quantitative understanding of the interaction between the genome and the proteome is necessary.

### SUMMARY OF THE INVENTION

It is one possible object of the invention to improve the identification of proteins that are suitable as a target for medicinal treatment of genetically related diseases or problems.

In order to identify pharmaceutical targets, at least one dependency or statistical correlation between the expression rates of different genes of a cell is ascertained by evaluating a multiplicity of gene expression patterns. In this case, inter alia, correlations of second or higher order are considered. The dependencies make it possible to infer causal relationships between different genes and the associated proteins. The regulatory network of the cell being studied can therefore be deduced from the dependencies.

In this way, it is possible to identify genes which most probably initiate regulatory cascades, or which are responsible for complex changes in the expression patterns, for example in the event of a genetically related disease.

The method therefore makes it possible to identify targets on a systematic basis. This is done by statistical modeling of the regulatory genetic network using a structure-learning causal network on the basis of gene expression patterns.

The described method does not rely on information as a function of time, and it can therefore be applied to a wide basis of gene expression measurements.

The described method is usually carried out with the aid of a computer.

The method and system are particularly suitable for supplementing high throughput drug discovery methods in biotechnology. Another application relates to the field of assisting tumor diagnosis and tumor treatment. It is possible to study regulatory relationships both in the human body and in any other living being, whether animal or vegetable, bacterium or another cell.

The individual measurements of the gene expression patterns are in this case regarded as mutually independent. They represent random values which are produced by an unknown high-dimensional probability distribution. Complete characterization of the statistical structure, that is to say of the correlations of the gene expression rates, with the aid of the measured gene expression patterns is equivalent to estimating the composite high-dimensional probability distribution for these patterns. If a measurement involves determining the expression of 5,000 genes, then a 5,000-dimensional probability density needs to be estimated, which most generally entails great difficulties.

Causal networks assume that conditional independencies exist in the data. There is a conditional independency whenever two random variables are mutually independent under the condition that all the other random variables are kept constant, that is to say higher-order correlations via a multistage feedback loop between the two random variables are neglected. The full probability density can then be replaced by a product of lower-dimensional probability densities.

A particularly efficient way of deducing the correlations or dependencies between the individual random variables, that is to say the expression rates, of the high-dimensional probability distribution involves firstly assuming a set of independent random variables. Successively, the correlation which most reduces the error of the network for the explanation of new data (generalization error) is added to the network in each case. This means that those correlations for which the actually measured gene expression patterns have the highest probability under all conceivable probability distributions are assumed. This is continued until the generalization error can be further reduced only within a predetermined threshold.

One preferred, simple embodiment of the search strategies for the correlations is carried out with the aid of the following steps:

firstly, the single edge which minimizes the generalization error is looked for, that is to say the best first edge,

the best second edge is subsequently looked for,

etc., until the generalization error can no longer be improved significantly.

In this way, it is possible to deduce both the correlations between the random variables (expression rates) and also the shape of the high-dimensional probability distribution, at least qualitatively in the latter case. The deduction of the correlations between the random variables, with the possibility of representing these correlations with the aid of at least partially directed graphs, is referred to as structure learning, since the structure of the regulatory network is learnt during this.

When successively adding correlations, it is possible to employ existing knowledge about regulatory relationships. In this way, the deduction of the regulatory relationships can be made faster and more accurate.

This algorithm, which is very time-consuming, especially for high-dimensional data, can be accelerated decisively by fast, quasi-optimal search strategies for important dependencies. One known algorithm for this is the greedy algorithm (T. H. Cormen, C. E Leiserson, R. L. Rivest, C. Stein: “Introduction to Algorithms”, 2nd edition McGraw-Hill Columbus, Ohio (2001)).

By artificial modification of individual gene expression rates, the most probable resulting gene expression pattern can be predicted from the structure of the regulatory network, that is to say of the high-dimensional probability distribution, calculated from the previously available data. This can be compared with measurements of diseased tissue (for example tumor tissue). In this way, it is possible to infer the gene group originally lying at the cause of a pathologically modified cellular function, or possibly the single gene lying at the cause, and to identify the associated protein as the target of a medicinal treatment.

### BRIEF DESCRIPTION OF THE DRAWINGS

These and other objects and advantages of the present invention will become more apparent and more readily appreciated from the following description of the preferred embodiments, taken in conjunction with the accompanying drawings of which:

FIG. 1 schematically shows the regulatory processes which determine the expression pattern of a cell;

FIG. 2 shows a directed acyclic graph; and

FIG. 3 illustrates ways of determining the direction of edges in a directed acyclic graph.

### DETAILED DESCRIPTION OF THE PREFERRED EMBODIMENT

Reference will now be made in detail to the preferred embodiments of the present invention, examples of which are illustrated in the accompanying drawings, wherein like reference numerals refer to like elements throughout.

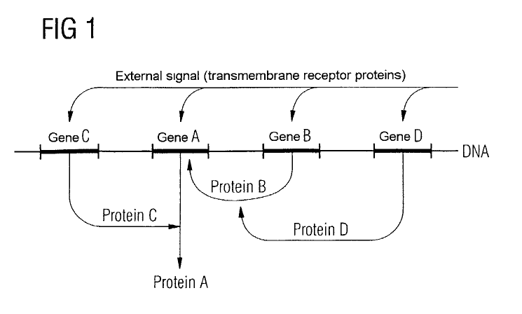

FIG. 1 shows the most important interactions between genes and proteins of a DNA segment. The interactions are used as the basis for describing the genomic regulatory network.

The upper part of FIG. 1 schematically indicates how an external signal acting on the cell from outside—for instance in the scope of intercellular communication—which is picked up for example by a transmembrane receptor protein (for example by a calcium channel) and is transmitted into the interior of the cell in a suitable way, triggers the production of the genes A, B, C and D of the DNA segment.

It is therefore in principle also possible to influence the expression rate of individual genes of a cell from outside the cells by the method.

The term “gene” denotes a not necessarily continuous segment of the DNA which contains the genetic code for a protein, or alternatively for a group of proteins.

The process for production of a protein from a gene, for example protein A on the basis of gene A in FIG. 1, is referred to as expression of this gene. The conversion of the DNA code of the gene into the chain of amino acids of the protein is referred to as translation. The rate at which protein A is produced in a given context is known as its expression rate.

Not all the genes are expressed in a cell. Rather, various cell types differ in terms of their gene expression pattern. This is often also true of the difference between diseased and healthy cells.

The expression pattern of a cell is determined by the regulatory processes schematically represented in FIG. 1. The regulatory processes are essentially determined by a few important interactions between proteins and genes, as well as of the proteins between one another.

For instance, the expression rate of a gene A may be regulated, that is to say increased, decreased or brought to a stop, by the presence of another protein B. In this example, protein B has a regulatory effect on gene A, or protein A. Regulatory proteins may, for example, be constituted by the protein units of activator complexes. Regulatory proteins may also act simultaneously on many target genes.

A second type of interaction involves the post-translational modification of proteins, that is to say the modification of proteins after translation. As a rule, post-translational modification of a protein takes place immediately after the end of translation, that is to say before the protein becomes active in the cell. For example, many proteins are phosphorylated or glycolyzed by special enzymes, that is to say the target protein is brought into its functional state, or put into a state in which it is no longer active, by adding or removing chemical groups. Post-translational modification may also functionally switch a protein on or off, possibly temporarily.

In FIG. 1, protein A is a so-called effector protein, that is to say it acts within the cell on other substances, and not directly on the genome or proteome. In FIG. 1, protein C hence modifies the function of the effector protein A through post-translational modification.

Protein B is a regulatory protein, since it determines the expression rate of protein A, by interacting with that DNA segment which contains gene A. Protein D hence modifies the function of a regulatory protein (protein B) through post-translational modification.

The nucleic acid sequence of human DNA is substantially known. The genes coded by the DNA are also being identified to an increasing extent. Knowledge about the proteome, including proteins possibly modified post-translationally by interaction between the proteins, is not so complete. Nevertheless, recent sequencing and high throughput screening methods are making rapid identification of further genes and proteins possible.

Another important step in the clarification of the expression patterns of a cell has come about with the development of high throughput hybridization techniques. In these methods, the expression rate of many 100 different genes are tested simultaneously on a so-called microarray. With the aid of these methods, it is possible to determine the gene expression pattern of a cell.

To that end, as a rule, the mRNA (messenger RNA) synthesized in the cell is determined. mRNA is an intermediate product during translation of the gene into the protein. mRNA is hence a precursor during formation of the protein. The cell to be studied is firstly isolated. It is subsequently broken up. By suitable purification steps, the mRNA from the cell is isolated. The mRNA is then transcribed by reverse transcriptase into cDNA (complementary DNA). The latter is amplified, as a rule by using linear PCR (polymerase chain reaction). The cDNA obtained in this way is qualitatively or quantitatively analyzed with the aid of suitable microarrays, for example DNA chips. With modern microarrays, the expression rates of 5,000 or more genes can be analyzed simultaneously.

On the basis of these improved techniques, extensive knowledge has by now become available about the human genome and proteome, as well as about the interactions between proteins and genes, and of proteins with one another.

Some mathematical terms needed for clarification of the regulatory network will firstly be introduced below.

The expression rates of the individual genes, which are determined from the measured gene expression patterns, are the random variables to be considered below. For gene i, the random variable representing the expression rate is denoted by Xᵢ. Values which it can take are denoted by xᵢ. The random vector, which consists of the expression rates of all k genes, is denoted by

$$X := ( \begin{matrix}{X}_{1} \\ … \\ {X}_{k}\end{matrix} ) = {( {X}_{1} , … , {X}_{k} )}^{T}$$

() ^{t} denotes transposition.

In order to ascertain the correlations between the expression rates, or the random variables, various moments of the random variables are considered.

The first moment of the random vector X, which is also referred to as the expectation value E, is defined by

EX :=(α₁, . . . ,αₖ)^{T} :=(EX₁, . . . , EXₖ)^{T} .

On the basis of known statistical considerations, the expectation value EXᵢ of the expression rates Xᵢ is estimated with the aid of the arithmetic mean of the observed expression rates xᵢ over n measurements of gene expression patterns:

$${E}^{( s )} {X}_{i} = \frac{1}{n} {\sum }_{m = 1}^{n} {x}_{im} ,$$

where xᵢₘ gives the expression rate determined for gene i in measurement m, and the superscript index (s) shows that an estimated value is involved.

The second moments are defined by

α₁ⱼ :=E(X₁·X_{J}).

Again, on the basis of known statistical considerations, the expectation value E(Xᵢ·Xⱼ) to be calculated for the second moment is estimated with the aid of the following equation:

$${E}^{( s )} ( {X}_{i} \cdot {X}_{j} ) = \frac{1}{n} {\sum }_{m = 1}^{n} {x}_{im} \cdot {x}_{jm} .$$

The second central moment is also referred to as the covariance. It is defined by

cov(X₁, Xⱼ):=μ₁ⱼ:=E([X₁−EXᵢ]·[X_{u-EX}ⱼ]).

Owing to the linearity of the expectation value, the following applies

cov(X₁, Xⱼ):=μ₁ⱼ:=E(X₁·Xⱼ)−EX₁·EXⱼ=αᵢⱼ−αᵢ·αⱼ.

The covariance is estimated in a known way by

$${cov}^{( s )} ( {X}_{i} , {X}_{j} ) = \frac{1}{n - 1} {\sum }_{m = 1}^{n} ( {x}_{im} - {E}^{( s )} {X}_{i} ) \cdot ( {x}_{jm} - {E}^{( s )} {X}_{j} ) .$$

The μᵢᵢ are precisely the variances of the individual expression rates Xᵢ:

σ₁² :=μₙ .

They are estimated in a known way using

$${σ}_{i}^{( s ) 2} = {μ}_{u}^{( s )} = \frac{1}{n - 1} {\sum }_{m = 1}^{n} {( {x}_{im} - {E}^{( s )} {X}_{i} )}^{2} .$$

The k×k matrix

cov(X, X):=E([X−EX]·[X−EX]^{T})=E(X·X^{T})−EX·EX^{T}

is referred to as the covariance matrix of X.

The correlation of the random variables Xᵢ and Xⱼ is often determined with the aid of the (second-order) correlation coefficient. This is defined by

$${ρ}_{ij} := \frac{cov ( {X}_{i} , {X}_{j} )}{{σ}_{i} \cdot {σ}_{j}} .$$

It lies between −1 and +1. It can likewise be estimated by using the indicated estimates for the covariance and the variance. A vanishing correlation coefficient points to the absence of regulatory relationships. A correlation coefficient differing significantly from zero points to a statistical and therefore regulatory dependency.

The above definitions can be generalized to third, fourth and any higher moments. In particular, the third moment is defined by

αᵢⱼₖ :=E(Xᵢ·Xⱼ·Xₖ).

The third central moment is defined by

μᵢⱼₖ :=E([Xᵢ−EXⱼ]·[Xⱼ−EXⱼ]·[Xₖ−EX_{k]).}

It is estimated in a known way by

$${μ}_{ijk}^{( s )} = \frac{1}{n - 2} {\sum }_{m = 1}^{n} ( {x}_{im} - {E}^{( s )} {X}_{i} ) \cdot ( {x}_{jm} - {E}^{( s )} {X}_{j} ) \cdot ( {x}_{km} - {E}^{( s )} {X}_{k} ) .$$

The correlation of the random variables Xᵢ, Xⱼ and Xₖ can likewise be determined with the aid of the third-order correlation coefficient. This is defined by

$${ρ}_{ijk} := \frac{{μ}_{ijk}}{{σ}_{i} \cdot {σ}_{j} \cdot {σ}_{k}} .$$

It likewise lies between −1 and +1, and can be estimated in the same way as the second-order correlation coefficient.

In an exemplary embodiment, the presence of regulatory dependencies is ascertained by testing the correlation coefficients in respect of whether they differ significantly from zero. Statistically speaking, the hypothesis that the correlation coefficient vanishes is tested. This can be done with the aid of various known statistical test methods. One method is, for example, described in Bronstein-Semendjajew: “Taschenbuch der Mathematik” (handbook of mathematics), Verlag Harri Deutsch, 22nd edition, 1985, p. 693.

The described methods generally have the purpose of clarifying statistical dependencies or independencies, and thereby extracting the network of influences from the data.

If protein B regulates gene A and there are no other regulatory phenomena, then this property is expressed in a statistical correlation or anti-correlation of the two expression rates over various measurements (second-order statistical dependency or correlation).

The presence of a metaregulator such as protein D in FIG. 1, however, is expressed in a third-order statistical dependency, that is to say in a non-vanishing third-order correlation coefficient.

In a cell, there are many partially still unknown regulatory feedback loops, the existence of which is expressed in complex statistical relationships between expression rates.

Correlations are often represented by directed graphs between random variables (see, for example, David Edwards: “Introduction to Graphical Modeling”, Springer Texts in Statistics, Springer Verlag, 1995). Such models are therefore also referred to as graphical models.

The high-dimensional probability distribution for the random variables

$$X = ( \begin{matrix}{X}_{1} \\ … \\ {X}_{k}\end{matrix} ) = {( {X}_{1} , … , {X}_{k} )}^{T}$$

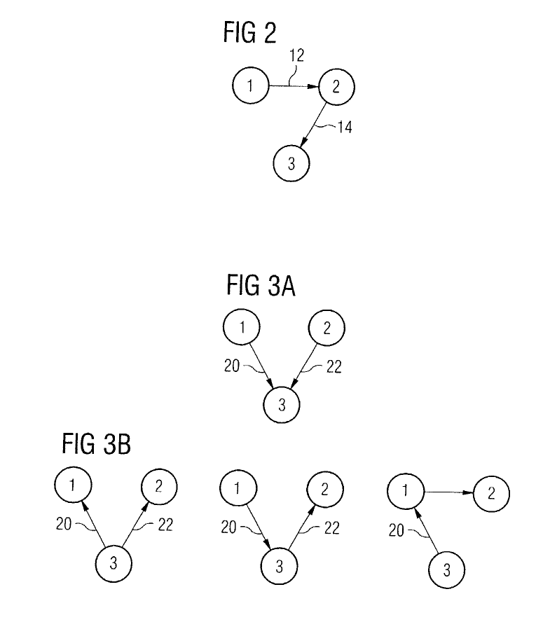

can be represented with the aid of a network or graph G, as shown in FIG. 2 for a simple example. The nodes 1, 2 and 3 correspond in this case to random variables X₁,X₂ , and X₃. In the scope of the statistical modeling of regulatory relationships in the genome, the random variables are identified with the expression rates.

In graph G according to FIG. 2, dependencies are represented by directed edges. In this case, the dependency of random variable X₂ on random variable X₁ is represented by a directed edge 12 from node 1 to node 2. The dependency of random variable X₃ on random variable X₂ is represented by a directed edge 14 from node 2 to node 3.

If a second-order correlation is established, then this is shown in the graph by an edge between two nodes, that is to say between two random variables. In general, it is not possible to ascertain the direction of this edge, that is to say which of the two random variables is the cause of the other. Only the simultaneous occurrence is observed. Therefore, it is also not in general possible to ascertain which of the two involved genes or proteins regulates the other.

In certain cases, however, the direction of an edge can be ascertained. FIG. 3A shows such a case. Three nodes 1, 2 and 3 are shown. Two edges are indicated between these three nodes, specifically the edge 20 between nodes 1 and 3 and the edge 22 between nodes 2 and 3. Both edges are directed toward node 3. In graph theory, such a case is generally referred to as a “collider”. Statistically, in such a constellation, a second-order correlation will be ascertained between nodes 1 and 3, that is to say the associated random variables, as well as a further second-order correlation between nodes 2 and 3. No third-order correlations, however, will be established since, for example, random variables 1 and 3 influence each other but without having an influence on random variable 2.

Put in terms of the regulatory interactions between genes or proteins, the graph according to FIG. 3A shows that gene 3 is regulated by genes or proteins 1 and 2, but not vice versa. If gene 1 is expressed, for example, then based on the model according to FIG. 3A gene 3 will also be expressed. This does not, however, imply that gene 2 will also be expressed. If two second-order correlations are found, one between node 1 and node 3 and the other between node 2 and node 3, then the edges cannot be directed differently since otherwise a third-order correlation would be shown (cf. FIG. 3B).

The situation is different in the case of FIG. 3B. FIG. 3B shows graphs which essentially correspond to the graph according to FIG. 3A, and which are to be read in a similar way. Only the edges and their directions are varied. All the graphs shown in FIG. 3B indicate exclusively a third-order correlation between nodes 1, 2 and 3, and they cannot be discriminated on the basis of correlation analysis.

In general, it is very difficult to deduce post-translational modifications on the basis of gene expression patterns. However, third-order correlations give at least an indication of such post-translational modifications.

The identification of the graph associated with a regulatory network will be explained in more detail below.

The common probability distribution of the random variables X₁, X₂ and X₃ in FIG. 2 can always be expressed by a product of conditional probabilities:

P(X₁,X₂, X₃)=P(X₃ |X₂, X₁)·P(X₂|X₁)·P(X₁).

In graph G according to FIG. 2, the conditional probabilities on the right-hand side are represented by directed edges. In this case, the conditional probability P(X₂|X₁) is represented by a directed edge 12 from node 1 to node 2. The conditional probability P(X₃|X₂,X₁) is represented by a directed edge 14 from node 2 to node 3. Such graphs G are referred to as directed acyclic graphs (DAGs). The graphs G are called acyclic since, in the mathematical model being considered, there is never a cyclic graph configuration in which, for example in FIG. 2, a directed edge also extends from node 3 to node 1, which would close a circle.

In the conditional probability P(X₃|X₂,X₁), the random variables X₁ and X₂ represent the so-called parents (Pa) of the random variable X₃, that is to say

Pa(X₃)={X₁, X₂}

In general, therefore, a high-dimensional probability distribution of the variables Xᵢ can be written as

$$P ( {X}_{1} , … , {X}_{k} ) = {\prod }_{i = 1}^{k} P ( {X}_{i} \vert Pa ( {X}_{i} ) ) .$$

In this case, Pa(Xᵢ) denotes the set of parents of the variable Xᵢ.

Statistical independencies can be determined in such a graph G by considering the parents of a random variable.

The structure of such a graph G is determined by comparison with obtained data, in the present case the measured expression patterns. The statistical problem can therefore be formulated in the following way: on the basis of a data record

$$D = ( \begin{matrix}{x}_{1}^{( 1 )} & {x}_{2}^{( 1 )} & ⋯ & {x}_{k}^{( 1 )} \\ {x}_{1}^{( 2 )} & {x}_{2}^{( 2 )} & ⋯ & {x}_{k}^{( 2 )} \\ ⋮ & ⋮ & & ⋮ \\ {x}_{1}^{( n )} & {x}_{2}^{( n )} & ⋯ & {x}_{k}^{( n )}\end{matrix} )$$

of n embodiments of the random variables (X₁, . . . , Xₖ), the graph G which best reproduces the data record D is looked for.

There are essentially two ways of deducing the structure of a graph G from the data D: The so-called “constrained based method” (R. Hofmann: “Lernen der Struktur nichtlinearer Abhängigkeiten mit graphischen Modellen” (learning the structure of nonlinear dependencies with graphical models), dissertation.de Berlin, 2000) and the so-called “score based method” (R. Hofmann: “Lernen der Struktur nichtlinearer Abhängigkeiten mit graphischen Modellen”, dissertation.de Berlin, 2000), which is perhaps preferred for implementation of the method and system.

The “constrained based method” attempts to deduce statistical dependencies or independencies from the data, in a similar way to that explained above in connection with the estimation of correlation coefficients.

The “score based method” searches through the space of the possible graphs and evaluates the correspondence between the graphs and the data with the aid of an evaluation function. The model that has the best value of the evaluation function is selected. Possible evaluation functions are the Bayes' measure (D. Heckerman: “A Bayesian Approach to learning causal networks”, Tech Report MSR-TR-95-04, Microsoft Research 1995), the MDL metric (see below) or the BIC evaluation function (G. Schwarz: “Estimating the dimension of a model”, The Annals of Statistics 6(2): 461-464 (1978)).

The evaluation function is the MDL metric. MDL stands for “minimum description length”. This evaluation function has the purpose of describing the data by a network, or a graph G, as accurately as possible with the fewest possible edges. The evaluation function that is used is written:

$$L ( G , D ) = log P ( G ) - n \cdot H ( G , D ) - \frac{1}{2} K \cdot log n .$$

In this case, logP(G) is the a priori probability (in the sense of a Bayes' evaluation) of the graph G being found. IogP(G) is assumed to be equal for all graphs G. It can therefore be ignored during the maximization of L.

n is the number of available measured data records.

$$H ( G , D ) = {\sum }_{i = 1}^{k} {\sum }_{e = 1}^{{E}_{t}} {\sum }_{l = 1}^{{r}_{i}} {\sum }_{j = 1}^{{q}_{ei}} - \frac{{N}_{ilej}}{n} log \frac{{N}_{ilej}}{{N}_{iej}}$$

reflects the conditional entropy of the graph G with respect to the data D.

In this case, as mentioned above, k is the number of random variables Xᵢ, or the number of nodes i. This means that summation is carried out over all the nodes.

Eᵢ is the number of direct parents of node i, that is to say the number of edges directed toward node i. This means that summation is additionally carried out over all the edges directed toward node i.

rᵢ is the number of possible (discrete or discretized) values xᵢ which the random variable Xᵢ can take, and therefore which the node i can take. This means that summation is carried out over all possible values of the random variable Xᵢ, or of the node i.

qₑᵢ is the number of possible (discrete or discretized) values xₑᵢ which the direct parent node e of node i, that is to say the random variable Xₑᵢ, can take. This means that summation is additionally carried out over all possible values of the random variable Xₑᵢ, or of the node e.

Nᵢₗₑⱼ is the number of data records in which node i has the value xₗ and the direct parent node e has the value xj, counted over all n data records. This means that the edge between nodes i and e is considered, and a count is made of how often the associated values xₗ and xⱼ occurred in the measured data records. The measured data converge here.

Lastly, the normalization is

$${N}_{iej} = {\sum }_{l = 1}^{{r}_{i}} {N}_{ilej} ,$$

that is to say summation is carried out over all values which the node i can assume.

The entropy is a non-negative measure of the uncertainty, which is a maximum when the uncertainty is a maximum, and which vanishes when there is complete knowledge.

K is given by:

$$K = {\sum }_{i = 1}^{k} {\sum }_{e = 1}^{{E}_{t}} {q}_{ei} \cdot ( {r}_{i} - 1 ) .$$

If the term “−1” in brackets is neglected, then K can be seen to reflect the number of all combinations of values, summed over all the edges. If the number of edges in a graph G is small, then as a rule K is also small, so that L is correspondingly larger. This last term on the right-hand side hence increases the value of L for graphs with few edges, so that it favors simple graphs. It is also referred to as evidence.

The evaluation function L corresponds approximately to the logarithm of the Bayes' probability for the graph G when the data D have been observed. It hence corresponds to a certain extent to the likelihood of the graph G. L is maximized, that is to say the graph G which maximizes the function L for the given data D is looked for.

A particularly efficient way of finding the edges of the graph G involves firstly assuming a set of independent random variables. Successively, the edge which most reduces the function L is added to the network in each case. This is continued until a minimum of L is achieved.

As already mentioned, this can be carried out in, simple type of embodiment with the aid of the following steps:

firstly, the single edge which minimizes L is looked for, that is to say the best first edge,

subsequently, the best second edge is looked for, that is to say the second edge which, in addition to the already existing first edge, most substantially minimizes L,

etc., until L can no longer be minimized further.

This algorithm, which is very time-consuming, especially for high-dimensional data, can be accelerated decisively by fast, quasi-optimal search strategies for important dependencies. One known algorithm for this is the greedy algorithm mentioned above.

In order to find not only local maxima of the graph structure, known algorithms such as simulated annealing or genetic algorithms may be used in combination with the algorithms described above, in order to look for the optimum graph.

Suitable targets can be identified from the regulatory network which has been deduced in such a way. For example, it can be seen in FIG. 1 that both gene A itself and also genes B, C, and D may be used as the target for influencing the concentration or efficacy of effector protein A.

The invention has been described in detail with particular reference to preferred embodiments thereof and examples, but it will be understood that variations and modifications can be effected within the spirit and scope of the invention.

1. A method of identifying pharmaceutical targets, comprising:
determining a plurality of gene expression patterns of a cell and for each gene expression pattern, determining expression rates for genes of the cell;
determining at least one dependency between the expression rates of different genes of the cell; and
deducing a regulatory network of the cell from the at least one dependency.

2. The method as claimed in claim 1, further comprising assuming that not all the expression rates of the genes of the cell are mutually dependent.

3. The method as claimed in the claim 1, wherein
a set of independent gene expression rates is taken as an initially assumption; and
modifying the initial assumption by successively assuming dependencies which most reduce errors in the gene expression rates.

4. The method as claimed in claim 1, wherein
a plurality of dependencies are determined, and
the dependencies are determined with the aid of a graph theory method.

5. The method as claimed in claim 1, further comprising;
artificially modifying the expression rate of at least one gene of the cell to produce a modified gene expression rate;
determining at least one modified gene expression pattern of the cell based on the modified gene expression rate; and
comparing the modified gene expression pattern with at least one gene expression pattern without modification.

6. The method as claimed in the claim 2, wherein
a set of independent gene expression rates is taken as an initially assumption; and
modifying the initial assumption by successively assuming dependencies which most reduce errors in the gene expression rates.

7. The method as claimed in claim 6, wherein
a plurality of dependencies are determined, and
the dependencies are determined with the aid of a graph theory method.

8. The method as claimed in claim 7, further comprising;
artificially modifying the expression rate of at least one gene of the cell to produce a modified gene expression rate;
determining at least one modified gene expression pattern of the cell based on the modified gene expression rate; and
comparing the modified gene expression pattern with at least one gene expression pattern without modification.

9. A system to identify pharmaceutical targets, comprising:
an expression unit to determine a plurality of gene expression patterns of a cell, the expression rate of the genes of the cell being determined in each case;
a correlation unit to determine at least one correlation between the expression rates of different genes of the cell; and
a network unit to deduce a regulatory network of the cell from the at least one correlation that has been determined.

10. A method of identifying pharmaceutical proteins, comprising:
determining a plurality of gene patterns for a cell;
determining the rate at which genes are expressed as proteins in the gene patterns;
determining dependencies between the expression rates of different genes;
developing a regulatory network for the cell, based on the dependencies, to describe interrelationships between the expression rates of different genes;
identifying a target gene expressing a target protein; and
using the regulatory network, identifying a protein which alters the expression rate of the target gene.
